(19) United States
(12) Patent Application Publication
(10) Pub. No.: US 2014/0265319 A1
(43) Pub. Date: Sep. 18, 2014

(54) ADAPTIVE CONNECTOR

(71) Applicant: SMITHS MEDICAL ASD, INC., Rockland, MA (US)

(72) Inventors: Geoff Clark, Lempster, NH (US); J. Michael Kennelly, Keene, NH (US)

(73) Assignee: SMITHS MEDICAL ASD, INC., Rockland, MA (US)

(21) Appl. No.: 14/203,740

(22) Filed: Mar. 11, 2014

Related U.S. Application Data
(60) Provisional application No. 61/783,009, filed on Mar. 14, 2013.

(51) Int. Cl. A61M 39/10

(52) U.S. Cl. CPC A61M 39/1011; A61M 2039/1027
USPC 285/330

(57) ABSTRACT

A connector has a first member with a distal portion non-removably coupled to a proximal portion of a second member. Respective through passages at the first and second members are in alignment to establish a through channel longitudinally along the connector. A seal in the connector prevents fluid leakage from the connector. A conventional fitting may be provided at one of the ends and a non-conventional fitting may be provided at the other end of the connector so that the connector may connect a device with a conventional fitting to a device with a non-conventional fitting. Once connected to the connector, the device with the conventional fitting is not removable from the connector. The first and second members are rotatable relative to each other about the longitudinal axis of the connector to enable rotational alignment of devices connected to the connector.

### FIELD OF THE INVENTION

The present invention relates to an connector or fitting adapter that is adapted to connect devices that have connector fittings that are not directly connectable to each other, and also act as an intermediary connector to devices with connector fittings.

### BACKGROUND OF THE INVENTION

To prevent mis-connection of a fluid line or a fluid store containing a particular medication to a needle and/or an infusion line, the prior art discloses the use of a two part connector with complementary configured opposing surfaces. Such two part connector is disclosed in U.S. Pat. No. 6,612,624 and its parent U.S. Pat. No. 6,402,207.

U.S. Pat. No. 7,497,484, assigned to the assignee of the instant application, discloses a three component connector or fitting adapter for connecting devices with different connector fittings that include a shroud, an inner shroud and a core. US application No. 11/342,620, filed Jan. 31, 2006, published as US 2007/0179454, also assigned to the assignee of the instant invention, discloses a fitting adapter that has a shroud and an adapter core fitted therein. In both the '484 patent and the '620 application, the adapter core has a through channel where fluid between the devices with different fittings flows. The respective disclosures of the '484 patent and the '620 application are incorporated by reference to the disclosure of the instant application.

Each fitting adapter of the '484 patent and the '620 application has a core through which fluid is directly conveyed between two devices each independently connected to one of the ends of the adapter. Due to possible mis-tolerance during the manufacturing of the components, friction that may result from the interaction between the components, and/or possible material dissimilarities of the mating to the components that cause the components to stick, there are instances where the rotation of one device connected to one end of the adapter may cause rotation of the other device connected to the other end of the adapter. As a result, instead of being able to remove a device from a particular end of the adapter, the device may actually be removed with the adapter attached thereto, as the adapter is disconnected at its other end from the other device.

### SUMMARY OF THE PRESENT INVENTION

The adaptive connector or fitting adapter (or simply connector or adapter) of the instant invention has a first member and a second member, with each of the members having a portion matable to each other. Respective coacting coupling mechanisms in each of the members ensure that the members, once matingly coupled to each other, remain non-removably connected to each other. The members have respective through passages that are aligned to be inline along a longitudinal axis, when the members are coupled together, so that the in alignment passages in combination establish a through channel wherethrough fluid may flow across the connector.

At the respective ends of the members not connected together there are provided different types of connector fittings or the same connector fitting. One of the fittings is a conventional fitting such as a female luer connector that is matable to a counterpart conventional fitting such as a male luer connector of one device. The other connector fitting is a non-conventional fitting, for example a male CorrectInject™ (CI) connector, that is matable only to a counterpart non-conventional fitting, i.e., a female CorrectInject™ (CI) connector, of another device. Both the male and female CI connectors are manufactured by the assignee of the instant application. An elastomeric seal such as a silicone O-ring is positioned at or proximate to the interface of the through passages of the two mated members to prevent fluid leakage from the adapter. An alternative embodiment of the inventive connector has both of its fittings being conventional fittings, i.e., luer fittings, so that the connector may be advantageously used as an intermediary to connect devices that have counterpart conventional connector fittings.

A ratchet mechanism is provided in the adapter to enable the members to rotate relative to each other in one direction but rotate together or in unison when either one of the members is rotated in a counter rotation, or a rotation that is opposite to the rotation that causes the members to rotate in unison. A device, such as a medical device including a syringe, a fluid line or a fluid store or bag, that has a counterpart conventional fitting is connectable to the conventional fitting of the one member. Once fully mated, the medical device is no longer removable from the adapter, as the rotation of the medical device in the direction opposite to that used to connect it to the adapter would merely cause the medical device to rotate with the one member of the adapter it is mated to, as the other member of the adapter remains stationary relative to the one member.

A second device such as a medical device including a needle or a fluid line for providing a medicament or infusate to the patient having a counterpart conventional or non-conventional fitting is matingly connectable to the conventional or non-conventional fitting at the other member of the adapter. Once fully mated, if the second medical device were turned in a counter direction, i.e., a rotation opposite to that used to connect it to the adapter, the second medical device likewise is not removable from the adapter, as the member to which it is connected would rotate together with the second medical device. The connector therefore enables the devices connected thereto to be rotated relative to each other to rotatably align to each other, if such rotational alignment is desired. However, the second medical device is removable from the adapter by a counter-rotational movement relative to the second member, if the housing of the second member, which forms the housing of the adapter, is fixedly held while the second medical device is turned.

The instant invention is therefore directed to a connector for connecting devices having conventional and/or non-conventional fittings to effect a fluid path between the fittings. The connector has a first member having a first through passage, a first coupling mechanism, a first distal portion with a distal end and a first proximal portion with a first proximal end including a first fitting matable with a counterpart first fitting of a first device. The connector further has a second member having a second distal portion with a second distal end and a second proximal portion connectable to the first distal portion. The second proximal portion has an inner diameter larger than the outer diameter of the first distal portion so that the first distal portion is inserted into or matable with the second proximal portion. A second through passage extends along the second distal portion and a second coupling mechanism is formed internally in the second proximal portion. A second distal end of the second member includes a conventional or non-conventional second fitting that is matable with a counterpart conventional or non-conventional fitting. When the first distal portion is matingly coupled to the second proximal portion, the first and second coupling mechanisms are non-removably engaged to each other with the first and second through passages in alignment to establish a fluid through channel across the first and second members. An O-ring is positioned in intimate contact with the first distal portion and the second proximal portion to effect a fluid tight seal for the connector. A ratchet mechanism in the adapter enables the selective rotation of the members of the connector.

Thus, the connector of the instant invention provides a through passage established by two members that are non-removably connected to each other but rotatable independent of each other when rotated in a given direction relative to each other, so as to enable the rotational alignment of the devices.

The present invention is also directed to a connector adapted to connect devices with conventional and/or non-conventional connector fittings that are not matable to each other. The connector has a first member having a first through passage, a first coupling mechanism and a first fitting at a first proximal end for mating with a counterpart first connector fitting of a first device. The connector further has a second member having a second through passage, a second coupling mechanism and a second fitting at a second distal end for mating with a counterpart second connector fitting of a second device. The first and second through passages are positioned in alignment along a longitudinal axis when the first and second members are connected to each other with the first and second coupling mechanisms engaged, so that the in-alignment first and second through passages effect a through channel across the first and second members to enable the exchange of fluid between the first and second devices when the first and second devices are mated to the first and second members, respectively. A seal may be provided in the connector to prevent fluid leak. A ratchet mechanism may be provided in the connector to enable the first and second members to rotate in unison when either of the members is rotated in one direction relative to the other, and each rotate independently of the other when either of the members is rotated in an opposite direction relative to the other. The one and other directions may also be referred to as the clockwise and counterclockwise rotation or direction.

The connector of the instant invention is also adapted to connect one device with a conventional connector fitting and an other device with a non-conventional connector fitting so that the one and other devices that otherwise are not connectable to each other may nonetheless be connected via the connector so that a fluid path may be established between the one and other devices. Conversely, the inventive connector prevents the inadvertent connection of a device, for example a medical syringe, to another device, for example a medical needle, that were not meant to be connected together, to thereby prevent mis-connection of the devices.

The invention is further directed to a method of making a connector for connecting two devices each with a connector fitting to enable the exchange of fluid between the devices that includes the steps of:
(a) providing a first member having a first through passage, a first coupling mechanism and a first fitting at a first proximal end for mating with a counterpart first connector fitting of a first device;
(b) providing a second member having a second through passage, a second coupling mechanism and a second fitting at a second distal end for mating with a counterpart second connector fitting of a second device;
(c) connecting the first and second members to each other;
(d) engaging the first and second coupling mechanisms together to prevent the first and second members from separating from each other; and
(e) positioning the first and second through passages in alignment with each other to effect a through channel across the first and second members.

The inventive method also includes the steps of providing a seal to prevent fluid leakage from the connector, and providing a ratchet mechanism in the connector to selectively enable the members of the connector to rotate in unison in one direction but rotate independently of each other when either of the members is rotated in an opposite direction relative to the other.

In an alternative embodiment, the connector may have conventional connector fittings at the respective ends of the members that are not coupled to each other so that the connector may act as a bridge for devices with conventional fittings where it may be advantageous to have one of the devices non-removably connected to the connector and the other of the devices removably connectable to the one device. This obviates the need to have the two devices connected directly to each other, as the connector of the instant invention provides a robust connector bridge for the devices in the event that it may not be a good idea to repeatedly connect/disconnect the conventional connector fitting of one of the devices to the conventional connector fitting of the other device.

### BRIEF DESCRIPTION OF THE FIGURES

The present invention will become apparent and the invention itself will be best understood with reference to the following description of the present invention taken in conjunction with the accompanying drawings, wherein:

FIG. 1 is a perspective view of the fitting adapter or adaptive connector of the instant invention;

FIG. 2 is another perspective view of the fitting adapter of the instant invention;

FIG. 3A is a perspective view of the housing or first member of the fitting adapter of the instant invention;

FIG. 3B is an end view of the member of FIG. 3A;

FIG. 3C is a view looking into the member of FIG. 3A;

FIG. 4A is a perspective view of the other or second member of the fitting adapter;

FIG. 4B is a side view of the FIG. 4A member;

FIG. 4C is a plan view of the distal end of the FIG. 4A member;

FIG. 4D is a plan view of the proximal end of the FIG. 4A member;

FIGS. 5A-5C are respective views of the O-ring seal of the instant invention;

FIG. 6 is a cross-sectional view showing the positioning of the respective portions of the first and second members of the inventive fitting adapter, and the positioning of the O-ring relative to the members;

FIG. 7 is a cross-sectional view showing the inventive fitting adapter, and the counterpart connectors that are mated to the conventional and non-conventional fittings of the inventive fitting adapter;

FIG. 8 is a cutaway view along section 8-8 of the fitting adapter;

FIG. 9 is a cross-sectional view of another embodiment of the inventive fitting adapter having conventional fittings at both of its ends; and

FIG. 10 is a cross-sectional view showing the coupling of the fittings of the connector of FIG. 9 with respective counterpart conventional connector fittings of two devices.

### DETAILED DESCRIPTION OF THE INVENTION

With reference to FIGS. 1 and 2, the fitting adapter or connector 2 of the instant invention is shown to include a first member 4 having a conventional female luer connector and a second member 6 having a non-conventional male CorrectInject™ (CI) connector non-removably mated to each other. For the embodiment shown in FIGS. 1 and 2, first member 4 may be referred to as a female luer member insofar as it has a proximal portion 4a with a proximal end 4b configured in the form of a conventional female luer fitting that includes a helical thread that may be formed as a non-continuous thread for ease of manufacture. The helical thread is represented by partial threads 4c and 4d. Thus configured, the conventional female luer of first member 4 is adapted to be connected to or matable with a counterpart conventional fitting, i.e., a conventional male luer connector, of a device such as for example a medical device that includes a syringe, a fluid line or a fluid store including a medicament storage bag or pump. Although the proximal portion (may also be referred to hereafter as the first proximal portion) of the first member 4 is designated 4a, it should be appreciated that the first proximal portion may in fact extend beyond a circumferential disklike flange 4e along the first member 4, per shown in FIG. 4B.

Further with reference to FIGS. 4A-4D, first member 4 is shown to be an elongate member having proximal portion 4a and a distal portion 4f (may also be referred to hereinafter as the first distal portion). For discussion purpose only, distal portion 4f and proximal portion 4a are deemed to meet at the second disklike flange 4g that circumferentially extends from the body of the first member, i.e., formed externally about the elongated member. Flange 4g has a chamferred surface 4g1 along its circumferential outer edge.

Further per shown in FIGS. 4A-4C, at the proximal portion 4f of the first member 4 are two pawls 4h each having a curved outer surface 4h1 that integrally curves upwardly from the outer wall of the elongated body to a pawl end 4h2. As best shown in FIG. 4A, there is a space between each of the pawls 4h and the main body of the first member 4 at proximal portion 4f. As a consequence, each of the pawls 4h may be flexibly bent downwards when its curved surface 4h1 encounters a force pressed thereagainst, for example by the ramped surfaces of the ramp stops of second member 6, as will be discussed in greater details below.

There is further provided at the distal portion 4f of the first member 4 a circumferential shoulder 4i that has a diameter smaller than either of flanges 4g and 4e. An extension 4j extends from shoulder 4i to the distal end 4k at the distal portion 4f of the first member 4. It is at extension 4j that an elastomeric seal such as the silicone O-ring shown in FIGS. 5A-5C may be positioned, as will be discussed further. As best shown in FIGS. 4A, 4C and 4D, a through passage 8, inline along the longitudinal axis 9 of member 4, extends throughout first member 4 between the opening 41 at proximal end 4b to the opening 4m (FIG. 4C) at distal end 4k. FIG. 4C shows through passage 8 in the end view of first member 4 from distal end 4k, and FIG. 4D shows through passage 8 in the end view from proximal end 4b.

The second member or the housing 6 of the fitting adapter of the instant invention is shown in greater detail in FIGS. 3A-3C. As shown in the perspective view of FIG. 3A, second member 6 has a proximal portion 6a (may be referred to hereinafter as the second proximal portion) and a distal portion 6b (may be referred to hereinafter as the second distal portion). As with the distal and proximal portions of the first member 4, there is no definitive demarcation point between the distal and proximal portions 6a and 6b for the second member 6. As will be further discussed with reference to FIGS. 6 and 7, a through passage 16 (or the second through passage) extends through distal portion 6b along second member 6. Distal portion 6b is shown to have an elongated extension 6c that has a circumferential outer surface made up of a number of ridges 6d and furrows 6f to assist a user to firmly grasp extension 6c. Distal portion 6b has a distal end 6g.

Second member 6 has at its proximal portion 6a an enlarged base or ring 6h that has a number of apertures or windows 6i which, for the embodiment shown in FIG. 3A, enable the forming of an internal non-continuous ring with a plurality of protrusions 6j. The non-continuous ring formed by the protrusions 6j may in practice be replaced by a continuous ring along the inner wall of ring 6h. It should moreover be appreciated that windows 6i are not necessary for second member 6, insofar as protrusions 6j may be molded as a part of an enclosed base that has no windows. Also, it should be noted that the dimension of 6h, in proportion to extension 6c, may be different than that shown inasmuch as the diameter of 6h may, in practice, be smaller or larger than that shown in FIGS. 3A-3C.

Each of projections 6j has a surface 6j1 that slopes downwards at an incline toward the interior of second member 6 from its inner wall 6k. The thus chamferred protrusions 6j enable disklike flange 4g, with counter chamferred surface 4g1 at its outer edge, to readily move past the protrusions 6j, when the first distal portion 4f is inserted into the second proximal portion 6a along the direction indicated by directional arrow 10. The back surfaces 6j2 of the respective protrusions, since they are flat, along with the flat back surface 4g2 of flange 4g, prevent first member 4 and second member 6 from separating once flange 4g moves past protrusions 6j and the respective back surfaces 4g2 of flange 4g and 6j2 of protrusions 6j are non-fixedly engaged to each other, per shown in FIGS. 6 and 7. For the embodiment shown, flange 4g and protrusions 6j may be considered as coacting coupling mechanisms. Flange 4g may also be referred to as the first or one coupling mechanism and protrusions 6j may also be referred to as the second or other coupling mechanism.

Base 6h of second member 6 also has an internal shoulder 6I whose inner wall is formed to have a plurality of ramp stops 6m. As best shown in the end view of FIG. 3C, each of the ramp stops 6m has a ramped surface 6m1 and an end stop 6m2 for coaction with the pawls 4h of first member 4. Together, the pawls 4h and the ramp stops 6m may be referred to as a ratchet mechanism for fitting adapter 2.

As best illustrated with FIG. 3C, if first member 4 were rotated, relative to second member 6, in the direction as indicated by directional arrow 12 (the clockwise direction), the ends 4h2 of pawls 4h would come into contact with the end stops 6m2 of the ramp stops 6m so that members 4 and 6 would rotate together in unison in the one direction, or the clockwise direction. On the other hand, if members 4 and 6 were rotated relative to each other in the direction as indicated by directional arrow 14 (the counterclockwise direction), the curved surfaces 4h1 of the pawls 4h would come into contact with the ramped surfaces 6m1 of the ramp stops 6m. As the pawls may be flexibly bent, albeit slight, due to the spatial distance separating the underside of their curved surfaces 4h1 and the body of member 4, the curved surfaces 4h1 and the ramped surfaces 6m1 would slide over each other when they come into contact. As a result, members 4 and 6 would rotate freely relative to each other when they are rotated along the other direction, or counterclockwise, relative to each other.

As shown from the distal end view of member 6 in FIG. 3B and the proximal end view in FIG. 3C, a through passage 16 extends through the distal portion 6b of second member 6. As further shown in FIGS. 3A and 3B, there is a wall portion 6o at member 6 that bridges the base 6h and extension 6c. Wall portion 6o has an internal circumference configured to accept the silicone O-ring 17 shown in FIGS. 5A-5C, or other similar elastomeric or rubber seals.

With reference to FIG. 3B, distal portion 6b is shown to have a nose cone 6p that extends along the distal portion 6b. See also FIGS. 6 and 7. FIG. 3B shows the notches 6q that form the entrance of the thread for accepting the nubbed hub, in particular the lugs of the hub, of a needle or medication infusion line, and the circumferential void 6r that extends into distal portion 6b for forming the non-conventional fitting (CI fitting) at the distal end 6g of second member 6.

FIG. 6 is a cross-sectional view of fitting adapter 2 showing the female luer fitting first member 4 and the male CI fitting second member 6 non-removably connected to each other. As shown, the first distal portion 4f of member 4 is inserted, per directional arrow 10, into the second proximal portion 6a of member 6 with flange 4g rotatably moveable relative to, but not removable from, protrusions 6j. One of the pawls 4h of member 4 is shown to be in movable contact relationship with a corresponding one of the ramp stops 6m of member 6. As further shown, flange 4e covers the opening at the proximal end 6n of base 6h. The diameter of flange 4e is such that there is sufficient tolerance between the circumferential edge of flange 4e and the inner wall of base 6h to ensure that members 4 and 6 are readily rotatable relative to each other without leaving too much space between the circumferential edge of flange 4e and the inner wall of base 6h, so that distal portion 4f remains firmly seated in proximal portion 6a, i.e., member flange 4e prevents rocking movement between members 4 and member 6. When distal portion 4f is fully inserted and seated in proximal portion 6a, flange 4e ensures that the through passage 8 at member 4 and the through passage 16 at the distal portion 6b of member 6 are aligned along the longitudinal axis 9. As a result, a through channel is established across the fitting adapter 2.

Although the distal end 4k of member 4 is in close proximity to the internal opening of passage 16 at distal portion 6b of member 6, to prevent leakage of fluid that passes through passages 8 and 16, an elastomeric seal such as a silicone O-ring 17 is placed in the space defined by the well 6o of member 6, so as to be in intimate contact with the outer wall of distal portion 4f of member 4 and the inner wall of well 6o, i.e., the inner wall of the proximal portion 6a of member 6. With O-ring 17 in place, a fluid tight seal is established between members 4 and 6 as fluid passes along the through channel formed by the inline passages 8 and 16. The internal thread 6s of the non-conventional CI fitting at distal portion 6b of member 6 is shown in the cross-sectional view of FIG. 6.

FIG. 7 shows the conventional female luer fitting at proximal end 4b of first member 4 threadingly mated to a counterpart conventional male luer fitting of a device 18 that may be a medical device such as a syringe, a catheter, an IV bag or other types of fluid medicament stores. The conventional male luer fitting is designated 20 in FIG. 7. Fitting adapter 2 is also shown to have matingly connected to its distal portion 6b, more specifically at distal end 6g that has the non-conventional fitting for example a male CI fitting, a counterpart non-conventional fitting for example a female CI fitting 22. The counterpart non-conventional fitting 22 is a part of a device 24 that may be a medical device such as for example a needle or a catheter connection through which medicament fluid may be supplied to a patient.

When fitting adapter 2 is matingly connected to the counterpart conventional fitting 20 by its conventional fitting at proximal end 4b, and matingly connected to the counterpart non-conventional fitting 22 by its non-conventional fitting at distal portion 6b, the medicament stored in device 18 is conveyable to a patient by means of device 24 via the through channel established by through passages 8 and 16 in fitting adapter 2. Device 18 has a passage 18a at its conventional male luer fitting 20 that is mated to the conventional female luer fitting at proximal end 4b of fitting adapter 2. Device 24 has a passage 24a at its counterpart non-conventional fitting 22 (female CI fitting) that is mated to the non-conventional fitting (male CI fitting) at distal end 16g of fitting adapter 2.

Device 18 is connectable to member 4 by rotating device 18, relative to member 4, in one direction, for example the clockwise direction designated by positional arrow 12, to mate the male luer fitting of device 18 to the female luer fitting of member 4. Once fully connected, additional rotation of device 18 in the same clockwise direction, relative to member 4 and member 6 being held fixedly, would cause members 4 and 6 to rotate in unison, as end 4h2 of at least one of the pawls 4h comes into contact with end stop 6m2 of at least one of the ramp stops 6m, as shown in FIG. 8. When device 18 is rotated in the opposite direction, for example the counterclockwise direction designated by directional arrow 14, relative to member 4, due to the curved surfaces 6m1 of the ramp stops 6m sliding over the curved surfaces 4h1 of pawls 4h, device 18 and member 4 are freely rotatable, in union, relative to member 6. Device 18 therefore is not removable from fitting adapter 2.

The counterpart non-conventional fitting of device 24 is connectable to the non-conventional fitting of member 6 of the adapter by rotating device 24 in the clockwise direction while member 6 is fixedly held, for example by the user's fingers holding elongate extension 6c. Once the non-conventional CI fittings are fully mated, additional rotation of device 24, relative to adapter 2, in the clockwise direction without fixedly holding in place member 6 would cause members 6 and 4 to rotate together in unison, as once again the end 4h2 of one of the pawls 4h comes into contact with one of the stops 6m2 of the plurality of ramp stops 6m to force the tandem rotation of members 4 and 6. This occurs when for example the user is holding device 18 (or device 24) while rotating device 24 (or device 18) in the clockwise direction.

When device 24 is rotated in the counterclockwise direction while device 18 is being fixedly held, device 24 is not removable from the distal portion 6b of member 6, as member 6 is rotated along with device 24, due to the plurality of pawls 4h and ramp stops 6m passing over each other by means of their respective curved surfaces 4h1 and ramped surfaces 6m1. However, device 24 is removable from fitting adapter 2 when extension 6c is fixedly held while device 24 is rotated in the counterclockwise direction.

Advantageously, given that the two members 4 and 6 have respective passages 8 and 16 that are separate from each other, the fluid passages are therefore independent from each other. As a result, the possibility that the rotational movement of one device attached to one end of the fitting adapter may cause an unintended rotation of the other device attached to the other end of the fitting adapter due to a single core having a single through passage is eliminated for the fitting adapter of the instant invention. Thus, subject to the interaction of the pawls with the ramp stops, the devices attached to the fitting adapter of the instant invention would rotate in unison in one direction and are freely rotatable relative to each other in the opposite direction, i.e., an other direction. Yet if the housing of the adapter were fixedly held, and the device with the counterpart non-conventional fitting is rotated in the counterclockwise direction relative to the adapter, the counterpart non-conventional fitting is removable from the non-conventional fitting of the adapter.

The fitting adapter may be manufactured cost effectively, in that members 4 and 6 each are molded from a medical plastics material such as polybutylene terephthalate. Moreover, the silicone O-ring 17 is an inexpensive component that may be an off shelf item. If appropriate, other elastomeric or rubber seals or rings may be used in place of the silicone O-ring.

To enable the user to readily determine that the fitting adapter of the instant invention is to be used to connect a medical device with a conventional fitting to another medical device with a non-conventional fitting, both members 4 and 6 may be color coded to have a specific color, for example yellow, to distinguish the adapter from other medical devices or components that may be in the vicinity of the fitting adapter, for example in the same tray or container as the fitting adapter. Alternatively, members 4 and 6 may have different colors to better distinguish the connector members, for example different colors for the conventional and non-conventional connector members.

Although discussed above as having a conventional fitting at distal end 4b and a non-conventional fitting at distal end 6g, it should be appreciated that the placement of those fittings may be reversed, for example the conventional luer fitting may be formed at the distal end 6g while the non-conventional CI fitting may be formed at the distal end 4b of the fitting adapter. Also, instead of the adapter having a female luer fitting and a male CI fitting, a yet further embodiment of the fitting adapter may have a conventional male luer fitting and a non-conventional female CI fitting. Furthermore, as discussed above, the dimension of base 6h, relative to the rest of member 6, may well be different from that shown. For example, base 6h may in actuality be reduced in dimension relative to the rest of member 6. With a reduction in the diameter of base 6h, flanges 4e and 4g likewise would have a smaller diameter, so that a portion of member 4 nonetheless is insertable into a corresponding portion of member 6 and non-removably attached thereto, as discussed above. It should moreover be noted that device 18 may be bonded to member 4, for example by gluing, so that the connector of the instant invention becomes a part of the medication dispensing device.

A variant of the connector of the instant invention is illustrated in FIGS. 9 and 10. The components that are the same and function the same as discussed above are labeled the same in FIGS. 9 and 10.

The adaptive connector 2′ shown in FIGS. 9 and 10 is substantially the same as connector 2 shown in the earlier drawings and discussed above, except for the distal portion 6b′ of member 6. For this embodiment of the inventive connector, the distal portion 6b′, and more particularly its internal thread 6s′, is configured as a conventional fitting, for example a luer fitting, that accepts a counterpart conventional connector fitting 22′ of a device 24′, such as for example a medical needle or an infusion line. As shown, the thread 6s′ formed at the inside wall of distal portion 6b′ is a conventional internal thread that is designed to accept the bosses or lugs 22a′ that are formed at the needle hub 22′ of the device 24′. Accordingly, the connector of FIGS. 9 and 10 is designed to have two conventional fittings, one at the proximal portion of member 4 and another at the distal portion 6b′ of member 6, so that the connector is adapted to act as a bridge to devices having conventional connector fittings, such as for example needles, syringes, pumps, catheters and infusion lines. For manufacturing purpose, the output orifice 8′ at the distal extension 4j of member 4 may be changed as shown so as to be different from the output orifice of member 4 of the embodiment shown in FIGS. 6 and 8.

In the case where connector 2′ has securely attached at its two ends the connector fittings of different devices, the fact that members 4 and 6 are rotatable relative to each other means that the devices connected to connector 2′ may be rotated about the longitudinal axis 9 of the connector to enable rotational alignment of the devices. Such rotational alignment attribute of the inventive connector advantageously also allows a device that is not meant to be repetitively connected and disconnected to be connected to one fitting of the inventive connector, so that the other fitting of the inventive connector may act as an intermediary connector to enable the device to be repeatedly connected to and disconnected from other devices. So, too, by being able to relatively rotate the devices that are coupled to the connector, the user may readily rotate or realign each of the respective connected devices, relative to the connector, without compromising the integrity of the fluid path established between the two devices, as the fluid passage along the inventive connector is a combination of two separate but in alignment through channels. One such exemplar use is the rotational repositioning or realignment of a manometer relative to a spinal needle that has been inserted into a patient to measure the pressure in the spinal fluid. To elaborate: the inventive connector may have connected to its one end a spinal needle (either 2 or 2′ depending on whether the coupling of the spinal needle to the connector is mating between conventional or non-conventional fittings), and it is desired to connect a manometer to the spinal needle to gauge the pressure in the spinal column. For this example, the manometer may comprise a 3-way stopcock with a long hollow plastic or glass cylinder or tubing inserted into the center port and its output port connected to the fitting at member 4 and its input port presumably connectable to a syringe or fluid line. To ensure that the spinal pressure is measured correctly, the tubing has to be at a vertical position relative to the needle. That the members of the inventive connector are rotatable relative to each other means that while the spinal needle is held stationary, the tubing may be rotated to its vertical orientation without compromising the security of the fluid path to thereby determine the fluid level in the vertical tubing, and hence the pressure in the spinal column.

Even though the rotational alignment of devices connected to the connector is discussed above with reference to conventional fittings and the connector shown in FIGS. 9 and 10, it should be appreciated that the devices coupled to the connector having conventional/non-conventional fittings shown in FIGS. 7 and 8 and discussed above can have the same rotational alignment as discussed with reference to FIGS. 9 and 10.

It should be appreciated that even though the embodiment of the inventive connector shown in FIGS. 7 and 8 and the variant of the connector shown in FIGS. 9 and 10 each show the distal portion of member 6 to have an internally threaded wall at 6b (6b′), that internally threaded wall (or internal thread) may not be necessary if the medical device that is to be mated to the connector has a hub that does not have any protrusions or lugs, such as 22a′ shown in FIG. 10, so that the device, by means of its hub, may simple slip-fit onto the nose cone 6p (6p′) of the connector. In other words, the inventive connector is adapted to connect to connector fittings that do not need to be lockingly mated to the connector. It is thus further envisioned that a connector of the instant invention may have a variant whereby the inner circumferential wall of the distal portion of member 6 that surrounds the tapered nose cone 6b (6b′) may comprise a non-threaded surface with tolerance sufficient to accept a slip-fit type connector fitting, be that slip-fit type connector fitting a conventional or non-conventional fitting.

The present invention is subject to many variations, modifications and changes in detail. Accordingly, it is intended that the instant invention be limited only by the spirit and scope of the hereto appended claims.

1. A connector to effect a fluid path between two devices, comprising:
a first member having a first through passage, a first coupling mechanism formed about the first member, a first distal portion with a first distal end and a first proximal portion with a first proximal end including a first fitting matable with a counterpart first connector fitting of one of the two devices;
a second member having a second distal portion with a second distal end and a second proximal portion connectable with the first distal portion of the first member, a second through passage extending along the second distal portion and a second coupling mechanism formed in the second proximal portion, the second distal end including a second fitting that is matable with a counterpart second connector fitting of other of the two devices;
wherein when the first distal portion is connected with the second proximal portion with the first and second coupling mechanisms engaged to each other, the first and second members are non-removably coupled to each other and the first and second through passages are in alignment with each other to establish a fluid through channel across the first and second members.

2. Connector of claim 1, wherein the first fitting of the first member comprises a conventional fitting and the first connector fitting of the one device comprises a counterpart conventional fitting matable to the conventional fitting of the first member; and
wherein the second fitting of the second member comprises a non-conventional fitting and the second connector fitting of the other device comprises a counterpart non-conventional fitting matable only with the non-conventional fitting, the non-conventional fitting and the counterpart non-conventional connector fitting only matable with each other.

3. Connector of claim 2, wherein the first fitting of the first member and the first connector fitting of the one device are connectable to each other when the one device is rotated in one direction relative to the connector, the one device not removable from the first member when rotated in an opposite direction relative to the connector once it is fully mated to the first proximal end of the first member; and
wherein the other device with the counterpart non-conventional fitting, once mated to the non-conventional fitting at the second member in one direction, is not removable from the connector when rotated in an opposite direction relative to the connector; and
wherein the other device is removable from the second member when the second distal portion is held fixed while the other device is rotated in the opposite direction relative to the connector.

4. Connector of claim 2, wherein the conventional fitting at the first proximal end comprises a conventional female luer fitting matable to a conventional male luer fitting of a medical device including at least any one of a syringe, a fluid line and a fluid bag.

5. Connector of claim 2, wherein the non-conventional fitting at the second distal end is not matable with a counterpart conventional fitting, the non-conventional fitting comprising a special connector having connected thereto any of a needle, a catheter and an infusion line.

6. Connector of claim 1, wherein the first connector fitting of the first device is matable to the first fitting at the first member when the first device is rotated in one direction relative to the connector, the first device not removable from the first member when rotated in an opposite direction relative to the connector once it is fully mated to the first member;
wherein the second connector fitting of the second device, once mated to the second fitting of the second member, is not removable from the connector when the second device is rotated in the opposite direction relative to the connector; and
wherein the second device is removable from the second member only when the second distal portion is held fixed while the second device is rotated in the opposite direction relative to the connector.

7. Connector of claim 1, wherein the connector has a longitudinal axis; and wherein after the first fitting and the first connector fitting are matingly coupled to each other and the second fitting and the second connector fitting are matingly coupled to each other so that the first and second devices each are connected to the connector, the devices are adapted to be moved into rotational alignment with each other as the devices are adapted to rotate relative to each other along one direction about the longitudinal axis of the connector.

8. Connector of claim 1, further comprising:
a seal positioned relative to the first distal portion and the second proximal portion after the first and second members are non-removably connected to each other to effect a fluid tight environment between the first and second members to prevent fluid leakage from the connector.

9. Connector of claim 1, further comprising at least one pawl at an outer wall of the first distal portion and at least one ramp stop at an inner wall of the second proximal portion, wherein the pawl and the ramp stop coact against each other so that the first and second members are rotatable in union when the first member is rotated in one direction relative to the second member, and wherein the pawl passes over the ramp stop so that the first and second members are rotatable relative to each other when the first member is rotated in an opposite direction relative to the second member.

10. Connector of claim 1, wherein the first member has a flange extending circumferentially about the first proximal portion, the flange substantially covering an opening at the second proximal end of the second proximal portion when the first distal portion and the second proximal portion are connected to each other.

11. Connector of claim 1, wherein the first coupling mechanism comprises a flange extending circumferentially from an outer wall of the first member and wherein the second coupling mechanism comprises at least one catch at an inner wall of the proximal portion, the flange and the catch non-removably engaged to each other when the first distal portion is inserted into the second proximal portion when the first member and the second member are coupled to each other, the engagement of the flange and catch preventing the first and second members from separating from each other.

12. Connector of claim 1, wherein the first member and the second member have different colors.

13. A connector for connecting devices with connector fittings, comprising:
a first member having a first through passage, a first coupling mechanism and a first fitting at a first proximal end for mating with a counterpart first connector fitting of a first device;
a second member having a second through passage, a second coupling mechanism and a second fitting at a second distal end for mating with a counterpart second connector fitting of a second device;
wherein the first and second through passages are positioned in alignment when the first and second members are connected to each other with the first and second coupling mechanisms engaged, the in alignment first and second through passages effecting a through channel across the first and second members to enable the exchange of fluid between the first and second devices when the first and second devices are mated to the first and second members, respectively, by the first fitting mating with the first counterpart connector fitting and the second fitting mating with the second connector fitting.

14. Connector of claim 13, wherein the first fitting of the first member is a conventional fitting and the second fitting of the second member is a non-conventional fitting; and
wherein the first connector fitting is a counterpart conventional fitting of the first fitting and the second connector fitting is a counterpart non-conventional fitting of the second fitting so that the first fitting is matable with the first connector fitting and the second fitting is matable only with the second connector fitting.

15. Connector of claim 13, wherein the first device with the counterpart conventional fitting is matable to the conventional fitting at the first member when the first device is rotated in one direction relative to the connector, the first device not removable from the first member when rotated in an opposite direction relative to the connector once it is fully mated to the first member;
wherein the second device with the counterpart non-conventional fitting, once mated to the non-conventional fitting of the second member, is not removable from the connector when the second device is rotated in the opposite direction relative to the connector; and
wherein the second device is removable from the second member only when the second distal portion is held fixed while the second device is rotated in the opposite direction relative to the connector.

16. Connector of claim 13, wherein the first connector fitting of the first device is matable to the first fitting at the first member when the first device is rotated in one direction relative to the connector, the first device not removable from the first member when rotated in an opposite direction relative to the connector once it is fully mated to the first member;
wherein the second connector fitting of the second device, once mated to the second fitting of the second member, is not removable from the connector when the second device is rotated in the opposite direction relative to the connector; and
wherein the second device is removable from the second member only when the second distal portion is held fixed while the second device is rotated in the opposite direction relative to the connector.

17. Connector of claim 13, wherein the connector has a longitudinal axis; and wherein after the first fitting and the first connector fitting are matingly coupled to each other and the second fitting and the second connector fitting are matingly coupled to each other so that the first and second devices each are connected to the connector, the devices are adapted to be moved into rotational alignment with each other as the devices are adapted to rotate relative to each other along one direction about the longitudinal axis of the connector.

18. Connector of claim 13, wherein the first through passage extends along a first distal portion of the first member and the second through passage extends along a second distal portion of the second member, the second member having a proximal portion into which the first distal portion is inserted when the first and second members are connected to each other, the connector further comprising:
a seal in intimate contact with both the second proximal portion and the first distal portion to prevent fluid leak from the connector.

19. Connector of claim 13, wherein the first coupling mechanism comprises a flange extending circumferentially from an outer wall of the first member and wherein the second coupling mechanism comprises at least one one-way catch at an inner wall of the second member, the flange and catch non-removably engaged to each other to prevent the first and second members from separating from each other once the first and second members are connected to each other.

20. Connector of claim 13, wherein a distal portion of the first member is inserted to a proximal portion of the second member when the first and second members are connected to each other, the first member having at least one pawl at the outer wall of the distal portion and the second member having at least one ramp stop at the inner wall of the proximal portion, wherein the pawl and the ramp stop coact against each other so that the first and second members are rotatable in union when the first member is rotated in one direction relative to the second member, and wherein the pawl passes over the ramp stop so that the first and second members are rotatable relative to each other when the first member is rotated in an opposite direction relative to the second member.

21. Connector of claim 13, wherein the first and second devices are medical devices, and wherein the device mated to the first member includes any of a syringe, a fluid line and a medicament fluid store; and wherein the device mated to the second member includes at least any of a needle, a fluid line and a catheter connector.

22. Connector of claim 13, wherein the first member and the second member have different colors.

23. A method of making a connector for connecting two devices each with a connector fitting, comprising the steps of:
(a) providing a first member having a first through passage, a first coupling mechanism and a first fitting at a first proximal end for mating with a counterpart first connector fitting of a first device;
(b) providing a second member having a second through passage, a second coupling mechanism and a second fitting at a second distal end for mating with a counterpart second connector fitting of a second device;
(c) connecting the first and second members to each other;
(d) engaging the first and second coupling mechanisms together to prevent the first and second members from separating from each other; and
(e) positioning the first and second through passages in alignment with each other to effect a through channel across the first and second members;
wherein the connector enables the exchange of fluid between the first and second devices when the first and second devices are mated to the first and second members, respectively.

24. Method of claim 23, wherein step (a) further comprises the step of:
configuring the first fitting as a conventional fitting and the first connector fitting as a counterpart conventional fitting matable with the conventional fitting of the first fitting, and
wherein step (b) further comprises the step of;
configuring the second fitting as a non-conventional fitting and the second connector fitting as a counterpart non-conventional fitting matable only with the non-conventional fitting.

25. Method of claim 20, wherein step (c) further comprises the steps of:
connecting the connector to the first device by mating the counterpart conventional fitting to the conventional fitting at the first member when the first device is rotated in one direction relative to the connector, the first device not removable from the first member when rotated in an opposite direction relative to the connector once it is fully mated to the first member;
connecting the connector to the second device by mating the counterpart non-conventional fitting of the second device with the non-conventional fitting of the second member;
wherein the second device is not removable from the connector when the second device is rotated in the opposite direction relative to the connector after the second device is connected to the connector; and
wherein the second device is removable from the second member only when the second distal portion is held fixed while the second device is rotated in the opposite direction relative to the connector.

26. Method of claim 20, wherein the connector has a longitudinal axis, and wherein after the first fitting and the first connector fitting are matingly coupled to each other and the second fitting and the second connector fitting are matingly coupled to each other so that the first and second devices each are connected to the connector, further comprising the step of:
rotating the devices relative to each other along one direction about the longitudinal axis of the connector to move the devices into rotational alignment with each other. as the devices are adapted to rotate relative to each other.

27. Method of claim 23, further comprising the steps of:
extending the first through passage along a first distal portion of the first member;
extending the second through passage along a second distal portion of the second member;
providing the second member with a proximal portion into which the first distal portion is inserted when the first and second members are connected to each other; and
providing a seal in intimate contact with both the second proximal portion and the first distal portion to prevent fluid leak from the connector.

28. Method of claim 23, wherein the first coupling mechanism comprises a flange extending circumferentially from an outer wall of the first member and wherein the second coupling mechanism comprises at least one one-way catch at an inner wall of the second member, and wherein step (d) comprises the step of:
non-removably engaging the flange and the catch to each other to prevent the first and second members from separating from each other once the first and second members are connected to each other.

29. Method of claim 23, wherein step (c) comprises the step of inserting a distal portion of the first member to a proximal portion of the second member; the method further comprising the steps of:
providing at least one pawl at the outer wall of the distal portion of the first member;
providing at least one ramp stop at the inner wall of the proximal portion of the second member;
wherein when the first member is rotated in one direction relative to the second member, the pawl and the ramp stop coact against each other so that the first and second members are rotatable in union; and
wherein when the first member is rotated in an opposite direction relative to the second member, the pawl passes over the ramp stop so that the first and second members are rotatable relative to each other.

30. Method of claim 23, further comprising the step of making the first member and the second member to have different colors.
